(19) United States
(12) Patent Application Publication
(10) Pub. No.: US 2017/0252006 A1
(43) Pub. Date: Sep. 7, 2017

(54) ULTRASONIC MEASUREMENT APPARATUS AND ULTRASONIC PROBE

(71) Applicant: SEIKO EPSON CORPORATION, Tokyo (JP)

(72) Inventor: Jiro TSURUNO, Okaya-shi (JP)

(73) Assignee: SEIKO EPSON CORPORATION, Tokyo (JP)

(21) Appl. No.: 15/438,298

(22) Filed: Feb. 21, 2017

(30) Foreign Application Priority Data
Mar. 4, 2016 (JP) 2016-041857

(51) Int. Cl. A61B 8/00; A61B 8/08; A61B 8/14

(52) U.S. Cl. CPC A61B 8/4461; A61B 8/14; A61B 8/4281; A61B 8/4483; A61B 8/5207

(57) ABSTRACT

An ultrasonic measurement apparatus includes: an ultrasonic element array for emitting an ultrasonic wave in a first direction; a columnar acoustic lens for changing the traveling direction of the ultrasonic wave emitted from the ultrasonic element array; and a linear guide for restricting a movement direction so that the acoustic lens and the ultrasonic element array move relatively each other in a second direction crossing the first direction and a column axis direction of the acoustic lens.

### BACKGROUND

1. Technical Field

The present invention relates to an ultrasonic measurement apparatus and an ultrasonic probe.

2. Related Art

An ultrasonic measurement apparatus for displaying ultrasonic waves emitted to a subject under examination using reflected waves, which are obtained by the reflection of the ultrasonic waves from the inside of the subject under examination, has been widely used. An insertion needle may be inserted into an organ, such as a blood vessel located inside the subject under examination using an ultrasonic measurement apparatus. JP-A-2003-334191 discloses an ultrasonic measurement apparatus including a guide unit for guiding an insertion needle. According to this, a hole for guiding the insertion needle is provided in the guide unit.

When inserting the insertion needle into the subject under examination, the operator performs an operation so that the insertion needle enters an ultrasonic wave emission range. As a result, an image of the insertion needle is displayed on the ultrasonic image. The operator checks whether or not the traveling direction of the insertion needle matches the target location while observing the ultrasonic image. The operator advances the insertion needle to the target location by adjusting the insertion angle of the insertion needle while observing the ultrasonic image.

The operator checks the insertion needle using an ultrasonic measurement apparatus. At this time, the operator adjusts a probe so that ultrasonic waves are emitted to the distal end of the insertion needle. Since the distal end of the insertion needle moves, the operator adjusts the probe while observing the display screen. When the probe disclosed in JP-A-2003-334191 is used, an operation to move the probe so that the insertion needle is imaged is performed. At this time, since the probe is operated in a state in which the probe is held by hand, the probe shakes. For this reason, the ultrasonic image shakes and the distal end of the insertion needle becomes not clear. As a result, the distal end of the insertion needle may not be able to be checked on the display screen. Therefore, an ultrasonic measurement apparatus capable of changing the traveling direction of ultrasonic waves with good operability has been desired.

### SUMMARY

An advantage of some aspects of the invention is to solve at least a part of the problems described above, and the invention can be implemented as the following forms or application examples.

### Application Example 1

An ultrasonic measurement apparatus according to this application example includes: an ultrasonic wave transmission unit that emits an ultrasonic wave in a first direction; a columnar acoustic lens that changes a traveling direction of the ultrasonic wave emitted from the ultrasonic wave transmission unit; and a movement restriction unit that restricts a movement direction such that the acoustic lens and the ultrasonic wave transmission unit move relatively each other in a second direction crossing the first direction and a column axis direction of the acoustic lens.

According to this application example, the ultrasonic measurement apparatus includes the ultrasonic wave transmission unit, the acoustic lens, and the movement restriction unit. The ultrasonic wave transmission unit emits an ultrasonic wave in the first direction. The acoustic lens is a columnar lens, and changes the traveling direction of the ultrasonic wave emitted from the ultrasonic wave transmission unit. The acoustic lens is a lens having a predetermined sectional shape extending in a predetermined direction, and the direction in which the predetermined sectional shape extends is referred to as a column axis direction. The second direction is a direction crossing the first direction and the column axis direction of the acoustic lens. In addition, when the acoustic lens and the ultrasonic wave transmission unit move relatively each other, the movement restriction unit restricts the movement direction so that the acoustic lens and the ultrasonic wave transmission unit move relatively each other in the second direction.

Accordingly, the second direction in which the acoustic lens and the ultrasonic wave transmission unit move relatively each other is a direction crossing the first direction. The second direction is a direction crossing the column axis direction of the acoustic lens. The acoustic lens is a lens whose thickness in the first direction changes corresponding to the position in the second direction. The surface of the acoustic lens on the first direction side has irregularities. In addition, the ultrasonic wave is input to the acoustic lens, and is output from the acoustic lens. The traveling direction of the ultrasonic wave output from the acoustic lens changes corresponding to the angle of the surface of the acoustic lens on the first direction side.

The acoustic lens and the ultrasonic wave transmission unit move relatively each other in the second direction. Accordingly, since the angle of the surface on the first direction side of the location where the ultrasonic wave is output from the acoustic lens is changed, the traveling direction of the ultrasonic wave output from the acoustic lens is changed. Therefore, the ultrasonic measurement apparatus can change the traveling direction of the ultrasonic wave. The traveling direction of the ultrasonic wave changes only in the second direction, but does not change in the column axis direction. Therefore, the ultrasonic measurement apparatus can change the traveling direction of the ultrasonic wave with good operability.

### Application Example 2

In the ultrasonic measurement apparatus according to the application example, the acoustic lens may be a convex lens, and a material of the acoustic lens may be a material in which a traveling speed of an ultrasonic wave is higher than that of an ultrasonic wave in a subject under examination.

According to this application example, the acoustic lens is a convex lens. In addition, the acoustic lens is used in contact with the subject under examination. The material of the acoustic lens is a material in which the traveling speed of the ultrasonic wave is higher than the traveling speed of the ultrasonic wave in the subject under examination. Therefore, the traveling speed of the ultrasonic wave is reduced when the ultrasonic wave enters the subject under examination from the acoustic lens. The acoustic lens is a convex lens, and the subject under examination functions as a concave lens. Accordingly, the ultrasonic measurement apparatus can emit the ultrasonic wave to a place wider than the width of the acoustic lens in the second direction.

### Application Example 3

In the ultrasonic measurement apparatus according to the application example, the acoustic lens may be a concave lens, and a material of the acoustic lens may be a material in which a traveling speed of an ultrasonic wave is lower than that of an ultrasonic wave in a subject under examination.

According to this application example, the acoustic lens is a concave lens. In addition, the acoustic lens is used in contact with the subject under examination. The material of the acoustic lens is a material in which the traveling speed of the ultrasonic wave is lower than the traveling speed of the ultrasonic wave in the subject under examination. Therefore, the traveling speed of the ultrasonic wave is increased when the ultrasonic wave enters the subject under examination from the acoustic lens. In addition, since the acoustic lens is a concave lens, the ultrasonic measurement apparatus can emit the ultrasonic wave to a place wider than the width of the acoustic lens in the second direction.

### Application Example 4

The ultrasonic measurement apparatus according to the application example further includes: a moving unit that moves the acoustic lens and the ultrasonic wave transmission unit relatively each other; a control unit that controls a relative position between the acoustic lens and the ultrasonic wave transmission unit; and an input unit that receives an input of the relative position.

According to this application example, the ultrasonic measurement apparatus further includes the moving unit, the control unit, and the input unit. The moving unit moves the acoustic lens and the ultrasonic wave transmission unit relatively each other. The control unit controls the relative position. The input unit receives an input of the relative position between the acoustic lens and the ultrasonic wave transmission unit that are moved relatively each other by the moving unit. The operator inputs the relative position through the input unit. The control unit controls the moving unit to move the acoustic lens and the ultrasonic wave transmission unit relatively each other. Therefore, the operator can perform an operation of moving the acoustic lens and the ultrasonic wave transmission unit relatively each other in the second direction.

### Application Example 5

The ultrasonic measurement apparatus according to the application example may further include: a moving unit that moves the acoustic lens and the ultrasonic wave transmission unit relatively each other; a control unit that controls the moving unit; an ultrasonic wave receiving unit that receives a reflected wave reflected by a subject under examination; an image forming unit that forms an ultrasonic image from data of the reflected wave; a determination unit that determines a subject of the ultrasonic image; and a display unit that displays the ultrasonic image. When the ultrasonic image does not include the subject, the control unit controls the moving unit such that the ultrasonic image includes the subject.

According to this application example, the ultrasonic measurement apparatus includes the moving unit, the control unit, the ultrasonic wave receiving unit, the image forming unit, and the determination unit in addition to the ultrasonic wave transmission unit, the acoustic lens, and the movement restriction unit. The moving unit moves the acoustic lens and the ultrasonic wave transmission unit relatively each other. The control unit controls the moving unit. The ultrasonic wave receiving unit receives a reflected wave reflected by the subject under examination. The image forming unit forms an ultrasonic image from the data of the reflected wave. The determination unit determines a subject of the ultrasonic image. The display unit displays an ultrasonic image.

The ultrasonic wave transmission unit emits an ultrasonic wave to the subject under examination, and the ultrasonic wave receiving unit receives a reflected wave that is reflected by the subject under examination. Then, the image forming unit forms an ultrasonic image from the data of the reflected wave, and the determination unit determines a subject of the ultrasonic image. At this time, when the ultrasonic image does not include a subject, the control unit controls the moving unit. Then, the ultrasonic image is made to include a subject. As a result, the operator can recognize the position of the subject by observing the ultrasonic image without controlling the moving unit.

### Application Example 6

In the ultrasonic measurement apparatus according to the application example, the subject may be a rod-shaped member having a distal end portion, and the control unit may detect the distal end portion and display the ultrasonic image including the distal end portion on the display unit.

According to this application example, the subject is a rod-shaped member having a distal end portion. The control unit detects the distal end portion. Then, the ultrasonic measurement apparatus displays an ultrasonic image having a subject in a place close to the distal end portion on the display unit. Accordingly, the operator can check the position of the distal end portion even when the distal end portion of the rod-shaped member moves.

### Application Example 7

In the ultrasonic measurement apparatus according to the application example, a liquid material may be disposed between the ultrasonic wave transmission unit and the acoustic lens.

According to this application example, the liquid material is disposed between the ultrasonic wave transmission unit and the acoustic lens. A change in the acoustic impedance between the ultrasonic wave transmission unit and the acoustic lens can be reduced by the liquid material. As a result, it is possible to suppress the reflection of the ultrasonic wave between the ultrasonic wave transmission unit and the acoustic lens.

### Application Example 8

According to this application example, an ultrasonic probe includes: an ultrasonic wave transmission unit that emits an ultrasonic wave in a first direction; a columnar acoustic lens that changes a traveling direction of the ultrasonic wave emitted from the ultrasonic wave transmission unit; and a movement restriction unit that restricts a movement direction such that the acoustic lens and the ultrasonic wave transmission unit move relatively each other in a second direction crossing the first direction and a column axis direction of the acoustic lens.

According to this application example, the ultrasonic probe includes the ultrasonic wave transmission unit, the acoustic lens, and the movement restriction unit. The ultrasonic wave transmission unit emits an ultrasonic wave in the first direction. The acoustic lens is a columnar lens, and changes the traveling direction of the ultrasonic wave emitted from the ultrasonic wave transmission unit. The acoustic lens is a lens having a predetermined sectional shape extending in a predetermined direction, and the direction in which the predetermined sectional shape extends is referred to as a column axis direction. The second direction is a direction crossing the first direction and the column axis direction of the acoustic lens. In addition, when the acoustic lens and the ultrasonic wave transmission unit move relatively each other, the movement restriction unit restricts the movement direction so that the acoustic lens and the ultrasonic wave transmission unit move relatively each other in the second direction.

Accordingly, the second direction in which the acoustic lens and the ultrasonic wave transmission unit move relatively each other is a direction crossing the first direction. The second direction is a direction crossing the column axis direction of the acoustic lens. The acoustic lens is a lens whose thickness in the first direction changes corresponding to the position in the second direction. The surface of the acoustic lens on the first direction side has irregularities. In addition, the ultrasonic wave is input to the acoustic lens, and is output from the acoustic lens. The traveling direction of the ultrasonic wave output from the acoustic lens changes corresponding to the angle of the surface of the acoustic lens on the first direction side.

The acoustic lens and the ultrasonic wave transmission unit move relatively each other in the second direction. Accordingly, since the angle of the surface on the first direction side of the location where the ultrasonic wave is output from the acoustic lens is changed, the traveling direction of the ultrasonic wave output from the acoustic lens is changed. Therefore, the ultrasonic probe can change the traveling direction of the ultrasonic wave. The traveling direction of the ultrasonic wave changes only in the second direction, but does not change in the column axis direction. Therefore, the ultrasonic probe can change the traveling direction of the ultrasonic wave with good operability.

### BRIEF DESCRIPTION OF THE DRAWINGS

The invention will be described with reference to the accompanying drawings, wherein like numbers reference like elements.

FIG. 1 is a schematic perspective view showing the configuration of an ultrasonic measurement apparatus according to a first embodiment.

FIG. 2 is a schematic side sectional view showing the structure of an ultrasonic probe.

FIG. 3 is a schematic side sectional view showing the structure of the ultrasonic probe.

FIG. 4 is a diagram showing an ultrasonic image.

FIG. 5 is a schematic side sectional view showing the structure of the ultrasonic probe.

FIG. 6 is a diagram showing an ultrasonic image.

FIG. 7 is a schematic side sectional view showing the structure of the ultrasonic probe.

FIG. 8 is a diagram showing an ultrasonic image.

FIG. 9 is a schematic side sectional view showing the structure of the ultrasonic probe.

FIG. 10 is a diagram showing an ultrasonic image.

FIG. 11 is an electrical control block diagram of an ultrasonic measurement apparatus.

FIG. 12 is a flowchart of an insertion needle insertion method.

FIG. 13 is an electrical control block diagram of an ultrasonic measurement apparatus according to a second embodiment.

FIG. 14 is a flowchart of the insertion needle insertion method.

FIG. 15 is a schematic side sectional view showing the structure of an ultrasonic probe according to a third embodiment.

FIG. 16 is a schematic side sectional view showing the structure of the ultrasonic probe.

FIG. 17 is a schematic side sectional view showing the structure of an ultrasonic probe according to a fourth embodiment.

FIG. 18 is a schematic side sectional view showing the structure of the ultrasonic probe.

FIG. 19 is a schematic side sectional view for explaining a method of detecting a blood vessel using ultrasonic waves according to a fifth embodiment.

FIG. 20 is a schematic side sectional view for explaining a method of detecting a blood vessel using ultrasonic waves.

### DESCRIPTION OF EXEMPLARY EMBODIMENTS

Hereinafter, embodiments will be described with reference to the accompanying diagram. In each diagram, the scale of each member is adjusted in order to have a recognizable size.

### First Embodiment

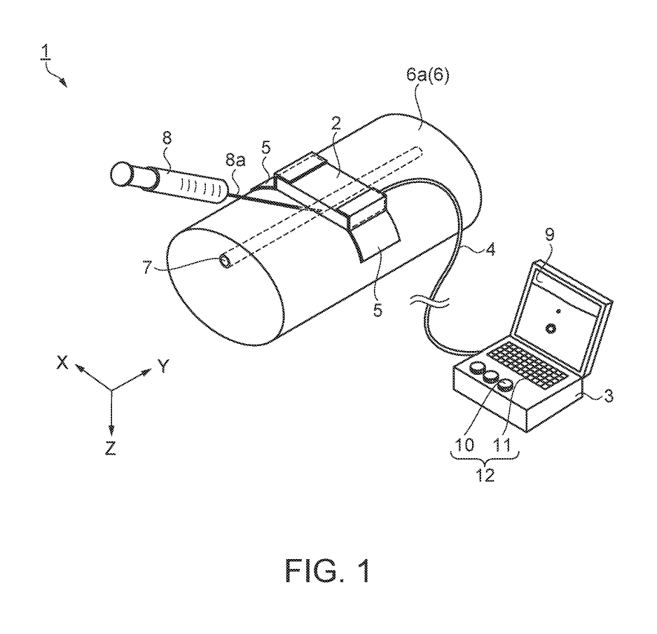

In the present embodiment, characteristic examples of an ultrasonic measurement apparatus and a method of inserting an insertion needle into a blood vessel using the ultrasonic measurement apparatus will be described with reference to the diagrams. The ultrasonic measurement apparatus according to the first embodiment will be described with reference to FIGS. 1 to 12. FIG. 1 is a schematic perspective view showing the configuration of the ultrasonic measurement apparatus. As shown in FIG. 1, the ultrasonic measurement apparatus 1 includes an ultrasonic probe 2 and a control device 3 as a control unit, and the ultrasonic probe 2 and the control device 3 are connected to each other by a wiring line 4.

The ultrasonic probe 2 is fixed to an arm 6a as a subject under examination of a human body 6 by a tape 5. In the arm 6a, a blood vessel 7 is present along the arm 6a. A direction in which the blood vessel 7 extends along the arm 6a is defined as a Y direction, and a direction toward the blood vessel 7 from the surface of the arm 6a is defined as a Z direction. A direction perpendicular to the Y and Z directions is defined as an X direction.

An operator inserts the insertion needle 8a as a subject of a syringe 8 into the blood vessel 7 of the arm 6a. In addition, the blood vessel 7 may be a vein or an artery. Then, the operator stops the movement of the insertion needle 8a when the operator inserts the insertion needle 8a into the blood vessel 7 and the distal end of the insertion needle 8a reaches the blood vessel 7. In this state, the operator injects medicine into the blood vessel 7. Alternatively, the operator sucks the blood in the blood vessel 7 into the syringe 8.

The ultrasonic probe 2 emits ultrasonic waves towards the distal end of the insertion needle 8a and the blood vessel 7. Then, reflected waves reflected by the distal end of the insertion needle 8a and the blood vessel 7 are received, and are converted into an electrical signal. The electrical signal is converted into a digital signal and is transmitted to the control device 3 through the wiring line 4. A display device 9 as a display unit is provided in the control device 3, and an ultrasonic image formed by the digital signal is displayed on the display device 9. The ultrasonic image is a sectional image of the arm 6a detected using the reflected waves of the ultrasonic waves.

An input device 12 as an input unit, such as a rotary knob 10 or a keyboard 11, is provided in the control device 3. The operator adjusts the traveling direction or the strength of ultrasonic waves emitted from the ultrasonic probe 2 by operating the input device 12. Then, the operator checks the position of the distal end of the insertion needle 8a with respect to the blood vessel 7 by observing the display device 9, and inserts the insertion needle 8a into the blood vessel 7. The operator stops the movement of the insertion needle 8a when the distal end of the insertion needle 8a enters the blood vessel 7. Then, injection of medicine, collection of blood, and the like are performed.

It is preferable to perform an operation by two persons. One person operates the input device 12, and the other person operates the syringe 8. The person who operates the input device 12 performs an operation so that the distal end of the insertion needle 8a is displayed on the display device 9. The person who operates the syringe 8 inserts the insertion needle 8a while observing the display device 9. Accordingly, the person who operates the syringe 8 can easily insert the insertion needle 8a into the blood vessel 7.

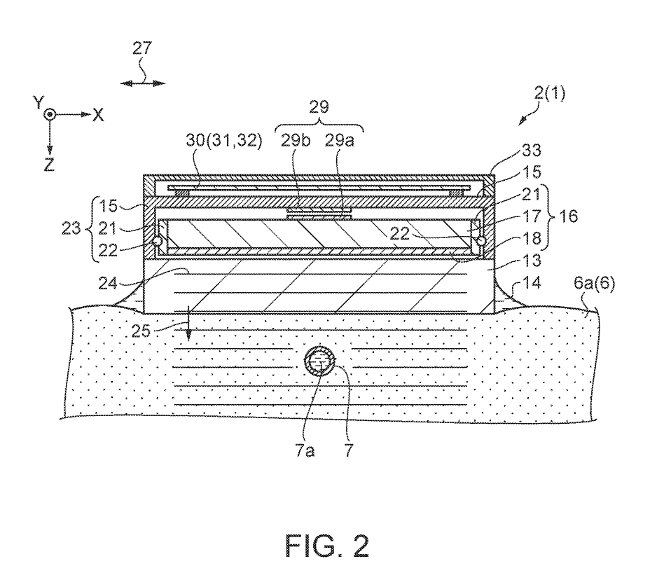

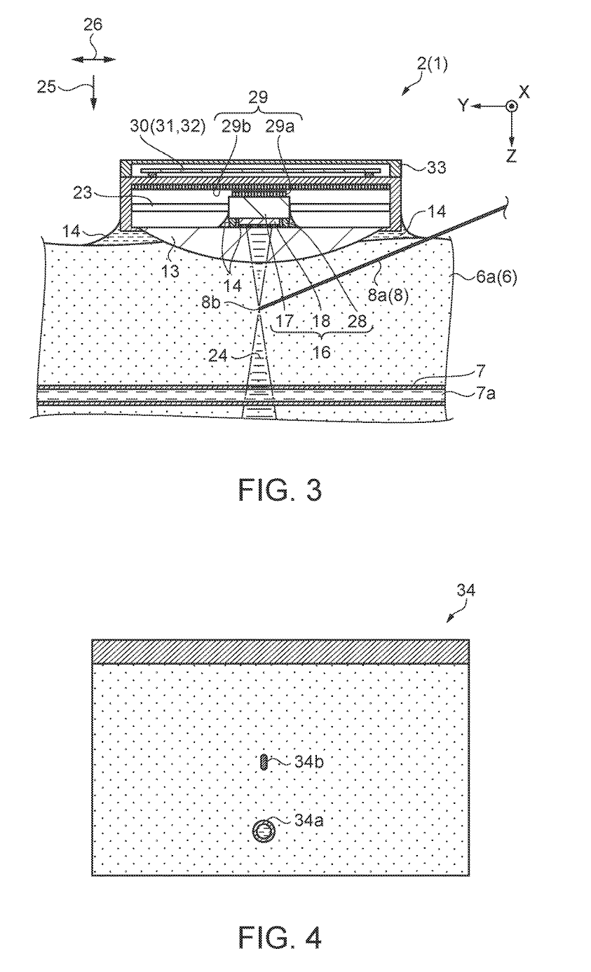

FIGS. 2 and 3 are schematic side sectional views showing the structure of an ultrasonic probe. FIG. 2 is a diagram viewed from the longitudinal direction of the blood vessel 7, and FIG. 3 is a diagram viewed from a direction perpendicular to the longitudinal direction of the blood vessel 7. In FIGS. 2 and 3, the tape 5 is omitted. As shown in FIGS. 2 and 3, the blood vessel 7 is located in the arm 6a, and blood 7a flows in the blood vessel 7.

The ultrasonic probe 2 includes an acoustic lens 13, and the acoustic lens 13 is pressed against the skin of the arm 6a. A gel 14 as a liquid material is applied on the surface of the skin. The gel 14 is disposed between the acoustic lens 13 and the arm 6a. The gel 14 adjusts the acoustic impedance between the acoustic lens 13 and the arm 6a. Due to the gel 14, it is difficult for an ultrasonic wave 24 to be reflected when entering the arm 6a from the acoustic lens 13. Therefore, the ultrasonic probe 2 can efficiently emit the ultrasonic wave 24 into the arm 6.

A bottomed rectangular tubular first support body 15 is provided on the −Z direction side of the acoustic lens 13. A moving body 16 that moves in the Y direction is provided in the first support body 15. The moving body 16 includes a substrate 17, and an ultrasonic element array 18 as an ultrasonic wave transmission unit and an ultrasonic wave receiving unit is provided on the surface of the substrate 17 on the +Z direction side.

A second support body 21 is provided on both surfaces of the substrate 17 on the +X and −X direction sides, and the substrate 17 is supported by being interposed between the second support bodies 21. A groove extending in the Y direction is provided on the +X-direction-side surface of the second support body 21 on the X direction side. A groove extending in the Y direction is provided on the −X-direction-side surface on the inner side of the first support body 15. A plurality of spheres 22 are provided between the grooves of the first support body 15 and the second support body 21. The −X-direction-side structure of the moving body 16 and the first support body 15 is the same as the +X-direction-side structure of the moving body 16 and the first support body 15. The first support body 15, the second support body 21, and the sphere 22 form a linear guide 23 as a movement restriction unit. In the linear guide 23, since the sphere 22 rolls, the frictional resistance at the time of movement is small.

In the ultrasonic element array 18, diaphragms are provided in a matrix on the silicon substrate. A piezoelectric element is provided on each diaphragm. An AC waveform is applied to the piezoelectric element. As a result, the piezoelectric element vibrates the diaphragm to emit the ultrasonic wave 24. An ultrasonic element is mainly formed by the diaphragm and the piezoelectric element. The emitted ultrasonic wave 24 travels through the arm 6a, and is reflected by the blood vessel 7 or the insertion needle 8a. The ultrasonic element array 18 receives the reflected wave of the ultrasonic wave 24. Some of the reflected ultrasonic waves vibrate the diaphragm to expand or contract the piezoelectric element. As a result, the piezoelectric element outputs a voltage corresponding to the reflected wave. The control device 3 forms an ultrasonic image using the voltage output from each piezoelectric element.

In the ultrasonic element array 18, one ultrasonic element may perform both transmission and reception of ultrasonic waves. In addition, an ultrasonic element with good ultrasonic wave transmission characteristics and an ultrasonic element with good ultrasonic wave reception characteristics may be arranged. Although the type of the piezoelectric element is not particularly limited, it is possible to use a piezoelectric element, such as a lead zirconate titanate (PZT) element or a polyvinylidene fluoride (PDVF) element. In the present embodiment, a PZT element is used as a piezoelectric element.

A direction in which the ultrasonic element array 18 emits the ultrasonic wave 24 is defined as a first direction 25. The first direction 25 is the Z direction. A direction in which the moving body 16 moves is defined as a second direction 26. The second direction 26 is the Y direction. The acoustic lens 13 has a shape obtained by cutting a cylinder along a plane parallel to the axial direction. The axial direction of the cylinder is defined as a column axis direction 27. The column axis direction 27 is the X direction.

An elastic packing 28 is provided on the surface of the substrate 17 on the +Z direction side. The packing 28 is provided so as to surround the ultrasonic element array 18. When the moving body 16 moves, the packing 28 slides on the acoustic lens 13. The gel 14 is provided between the substrate 17 and the acoustic lens 13, and the gel 14 is also surrounded by the packing 28. In addition, when the moving body 16 moves, the gel 14 also moves with the moving body 16. Therefore, the gel 14 is always located between the ultrasonic element array 18 and the acoustic lens 13.

A change in the acoustic impedance between the ultrasonic element array 18 and the acoustic lens 13 can be reduced by the gel 14. As a result, it is possible to suppress the reflection of the ultrasonic wave 24 between the ultrasonic element array 18 and the acoustic lens 13.

On the surface of the substrate 17 on the −Z direction side, a permanent magnet 29a is provided. In the permanent magnet 29a, an S pole and an N pole are alternately arranged in the Y direction so as to be magnetized at fine pitches. On the surface of the first support body 15 on the +Z direction side, an electromagnet 29b is provided. In the electromagnet 29b, coils are arranged side by side in the Y direction. The permanent magnet 29a and the electromagnet 29b form a linear motor 29 as a moving unit. By switching a current flowing through the coil in the linear motor 29, the S pole and the N pole are switched. In addition, the linear motor 29 moves the moving body 16 by applying a Lorentz force between the permanent magnet 29a and the electromagnet 29b.

In addition to the electromagnetic linear motor 29, it is possible to use a linear piezoelectric motor driven by a piezoelectric element, a linear resonance actuator that is moved by vibration, and the like.

A circuit board 30 is provided on the −Z direction side of the first support body 15. A motor driving circuit 31 for driving the linear motor 29 and a transducer driving circuit 32 for driving the ultrasonic element array 18 are mounted on the circuit board 30. The circuit board 30 is connected to the control device 3 by the wiring line 4. On the −Z direction side of the first support body 15, a housing 33 is provided so as to cover the circuit board 30. The housing 33 prevents the circuit board 30 from being short-circuited or from becoming dirty.

As shown in FIG. 2, the ultrasonic element array 18 has a shape that is long in the column axis direction 27. In addition, an ultrasonic image displayed on the display device 9 is an image of a plane passing through the column axis direction 27 and the first direction 25.

As shown in FIG. 3, the linear motor 29 moves the moving body 16 in the second direction 26. In other words, the linear motor 29 moves the acoustic lens 13 and the ultrasonic element array 18 relatively each other. When the moving body 16 and the ultrasonic element array 18 are located at the center in the second direction 26, the ultrasonic wave 24 travels in the first direction 25. Then, when the insertion needle 8a is located in the first direction 25 of the ultrasonic element array 18, some of the reflected waves of the ultrasonic waves 24 are input to the ultrasonic element array 18 from the insertion needle 8a.

FIG. 4 is a diagram showing an ultrasonic image. An ultrasonic image 34 is an image formed by the reflected wave of the ultrasonic wave 24 shown in FIG. 3. As shown in FIG. 4, a blood vessel image 34a showing the blood vessel 7 and an insertion needle image 34b showing the insertion needle 8a are displayed on the ultrasonic image 34. At this time, since the insertion needle 8a is a rod-shaped member and a distal end portion 8b, which is the distal end of the insertion needle 8a, is away from the blood vessel 7, an image is obtained in which the blood vessel image 34a and the insertion needle image 34b are separated from each other.

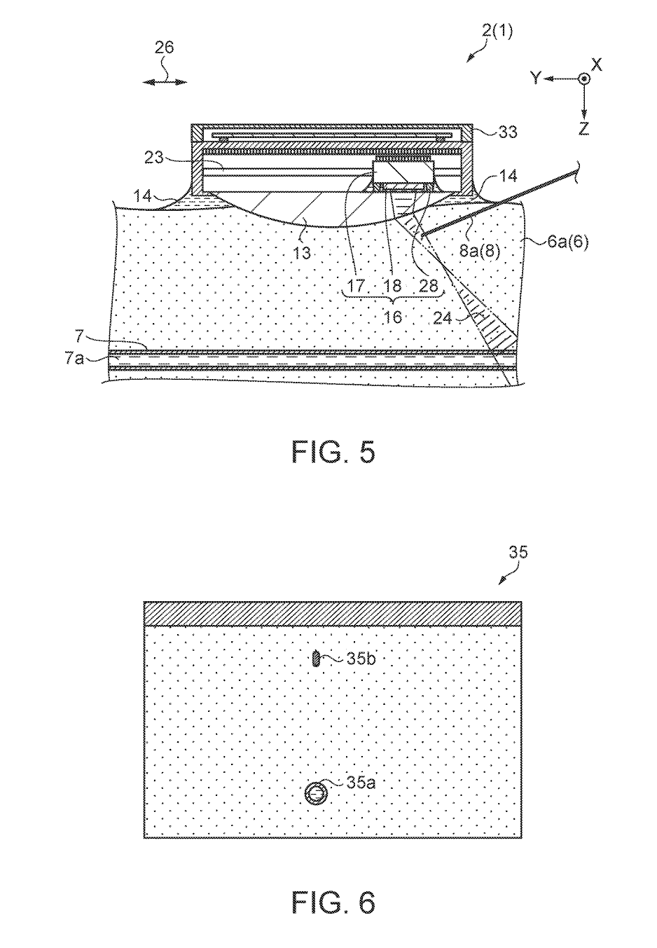

FIG. 5 is a schematic side sectional view showing the structure of the ultrasonic probe, and is a diagram when viewed from a direction perpendicular to the longitudinal direction of the blood vessel 7. As shown in FIG. 5, when the moving body 16 moves to the −Y direction side along the second direction 26, the ultrasonic wave 24 travels to the −Y direction side due to the acoustic lens 13.

The material of the acoustic lens 13 is a material in which the traveling speed of the ultrasonic wave 24 is higher than the traveling speed of the ultrasonic wave 24 in the arm 6a. The traveling speed of the ultrasonic wave 24 in the arm 6a is 1530 m/s. Nylon, polystyrene, and polyethylene can be used as materials of the acoustic lens 13. The traveling speed of the ultrasonic wave 24 in nylon is 2600 m/s, the traveling speed of the ultrasonic wave 24 in polystyrene is 2350 m/s, and the traveling speed of the ultrasonic wave 24 in polyethylene is 1900 m/s. In the present embodiment, for example, nylon is used as the material of the acoustic lens 13.

At this time, the traveling speed of the ultrasonic wave 24 is reduced when the ultrasonic wave 24 enters the arm 6a from the acoustic lens 13. The acoustic lens 13 is a convex lens, and the subject under examination functions as a concave lens. Accordingly, the ultrasonic probe 2 can emit the ultrasonic wave 24 to a place wider than the width of the acoustic lens 13 in the second direction 26. By moving the moving body 16 to the −Y direction side, the ultrasonic wave 24 is made to travel to the −Y direction side. Therefore, it is possible to detect the insertion needle 8a inserted from the −Y direction side.

FIG. 6 is a diagram showing an ultrasonic image. An ultrasonic image 35 is an image formed by the reflected wave of the ultrasonic wave 24 shown in FIG. 5. As shown in FIG. 6, a blood vessel image 35a showing the blood vessel 7 and an insertion needle image 35b showing the insertion needle 8a are displayed on the ultrasonic image 35. At this time, since the distal end portion 8b of the insertion needle 8a is away from the blood vessel 7, an image is obtained in which the blood vessel image 35a and the insertion needle image 35b are separated from each other. Then, it is possible to check that the insertion needle image 35b is located in a shallow place of the arm 6a.

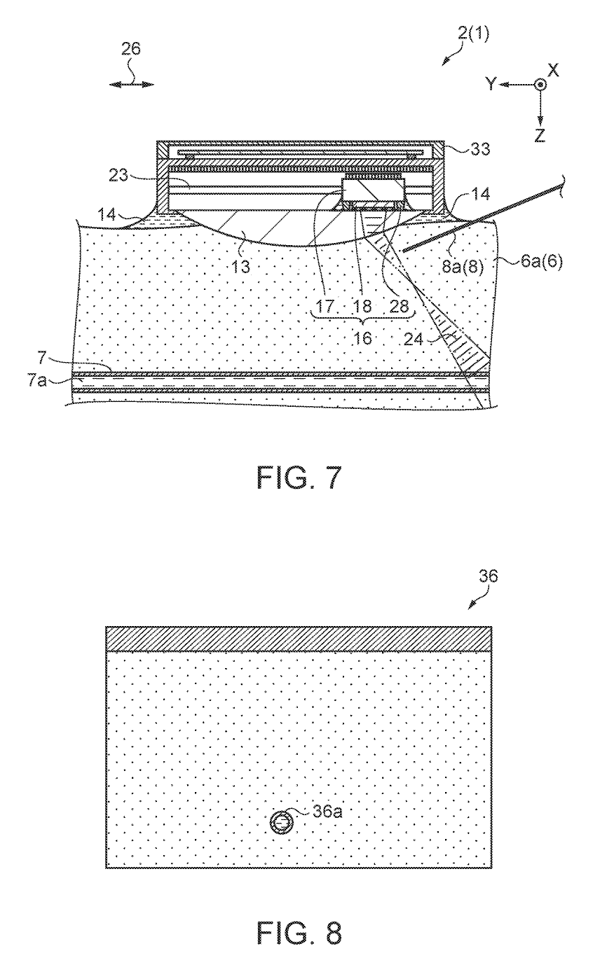

FIG. 7 is a schematic side sectional view showing the structure of the ultrasonic probe, and is a diagram when viewed from a direction perpendicular to the longitudinal direction of the blood vessel 7. As shown in FIG. 7, when the moving body 16 moves to the +Y direction side by a short distance along the second direction 26, the ultrasonic wave 24 passes the +Y direction side from the distal end portion 8b of the insertion needle 8a. Accordingly, there is no reflected wave from the insertion needle 8a.

FIG. 8 is a diagram showing an ultrasonic image. An ultrasonic image 36 is an image formed by the reflected wave of the ultrasonic wave 24 shown in FIG. 7. As shown in FIG. 8, a blood vessel image 36a showing the blood vessel 7 is displayed on the ultrasonic image 36. At this time, it is not possible to check an image corresponding to the insertion needle 8a. Since it is not possible to check the image corresponding to the insertion needle 8a even if the distance of the movement of the moving body 16 is short, it can be seen that the ultrasonic wave 24 has passed the vicinity of the distal end portion 8b of the insertion needle 8a. Therefore, the operator can recognize that the position of the insertion needle image 35b of the ultrasonic image 35 is close to the position of the distal end portion 8b of the insertion needle 8a.

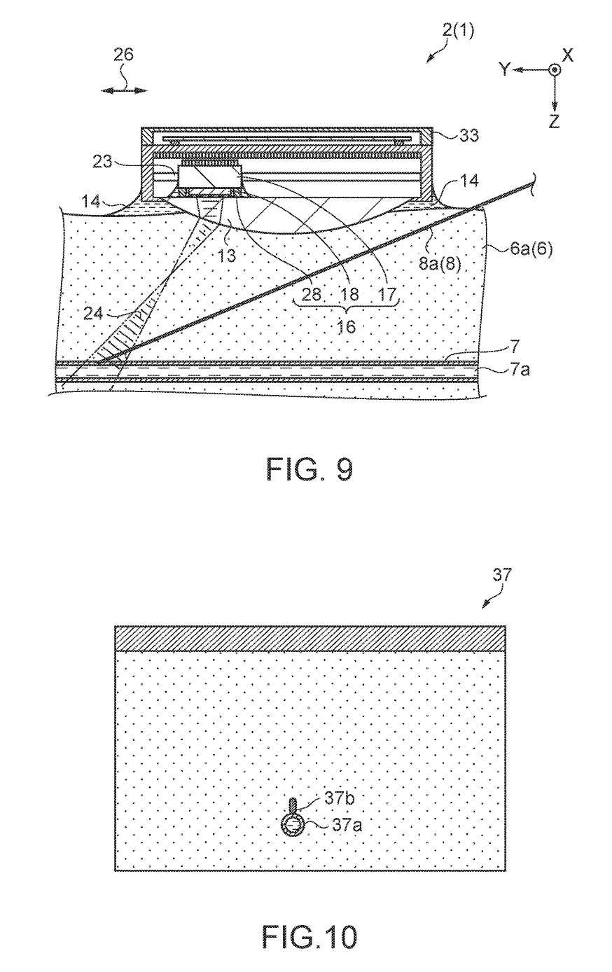

FIG. 9 is a schematic side sectional view showing the structure of the ultrasonic probe, and is a diagram when viewed from a direction perpendicular to the longitudinal direction of the blood vessel 7. As shown in FIG. 9, the insertion needle 8a is inserted in the +Y direction. At this time, the control device 3 moves the moving body 16 to the +Y direction side along the second direction 26. Then, since the ultrasonic wave 24 passes through the distal end portion 8b of the insertion needle 8a, the ultrasonic element array 18 detects reflected waves from the distal end portion 8b of the insertion needle 8a.

FIG. 10 is a diagram showing an ultrasonic image. An ultrasonic image 37 is an image formed by the reflected wave of the ultrasonic wave 24 shown in FIG. 9. As shown in FIG. 10, a blood vessel image 37a showing the blood vessel 7 and an insertion needle image 37b showing the insertion needle 8a are displayed on the ultrasonic image 37. At this time, since the distal end portion 8b of the insertion needle 8a is in contact with the blood vessel 7, an image is obtained in which the blood vessel image 37a and the insertion needle image 37b are in contact with each other. Then, the operator can check that the insertion needle 8a has reached the blood vessel 7.

Thus, the ultrasonic element array 18 emits the ultrasonic wave 24 in the first direction 25. The acoustic lens 13 is a columnar lens, and changes the traveling direction of the ultrasonic wave 24 emitted from the ultrasonic element array 18. The acoustic lens 13 is a lens having a predetermined semicircular sectional shape extending in the column axis direction 27. The second direction 26 is a direction crossing the first direction 25 and the column axis direction 27. The linear guide 23 restricts the movement direction so that the acoustic lens 13 and the ultrasonic element array 18 move relatively each other in the second direction 26.

The acoustic lens 13 is a lens whose thickness in the first direction 25 changes corresponding to the position of the second direction 26. The ultrasonic wave 24 is input to the acoustic lens 13, and is output from the acoustic lens 13. The traveling direction of the ultrasonic wave 24 output from the acoustic lens 13 changes corresponding to the angle of the surface of the acoustic lens on the +Z direction side.

The acoustic lens 13 and the ultrasonic element array 18 move relatively each other in the second direction 26. Then, when the ultrasonic wave 24 is output from the acoustic lens 13, the traveling direction of the ultrasonic wave 24 is changed. Therefore, the ultrasonic probe 2 can change the traveling direction of the ultrasonic wave 24. The traveling direction of the ultrasonic wave 24 changes only in the second direction 26, but does not change in the column axis direction 27. When the traveling direction of the ultrasonic wave 24 changes in the column axis direction 27, images of the ultrasonic images 34 to 37 shake in the horizontal direction. Accordingly, it is difficult to check the images. The ultrasonic measurement apparatus 1 can change the traveling direction of the ultrasonic wave 24 only in the second direction 26 with good operability. Accordingly, since the traveling direction of the ultrasonic wave 24 does not change in the column axis direction 27, it becomes easy to check the image by suppressing the shaking of the images of the ultrasonic images 34 to 37 in the horizontal direction.

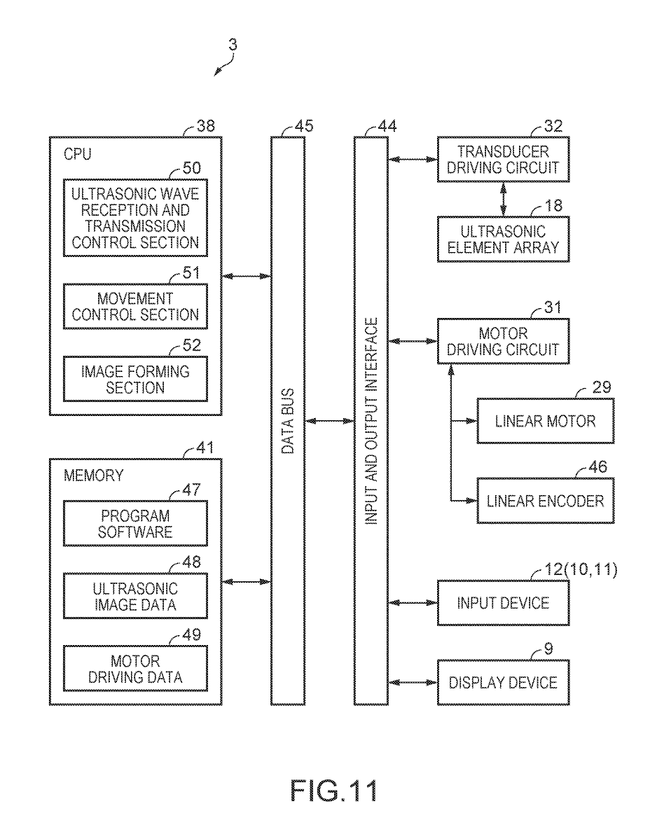

FIG. 11 is an electric control block diagram of the ultrasonic measurement apparatus. In FIG. 11, the ultrasonic measurement apparatus 1 includes the control device 3 that controls the operation of the ultrasonic measurement apparatus 1. In addition, the control device 3 includes a central processing unit (CPU 38) that performs various kinds of arithmetic processing as a processor and a memory 41 that stores various kinds of information. The transducer driving circuit 32, the motor driving circuit 31, the input device 12, and the display device 9 are also connected to the CPU 38 through an input and output interface 44 and a data bus 45.

The transducer driving circuit 32 is a device for driving the ultrasonic element array 18. The transducer driving circuit 32 receives an instruction signal from the CPU 38. Transducers are provided in the ultrasonic element array 18. The transducer driving circuit 32 sequentially vibrates the transducers at predetermined locations. The ultrasonic waves 24 are emitted at the locations where the transducers vibrate. The emitted ultrasonic waves 24 are reflected by the blood vessel 7 or the insertion needle 8a, and some of the ultrasonic waves 24 reach the ultrasonic element array 18. In the ultrasonic element array 18, transducers are vibrated by the received ultrasonic wave 24, and a voltage signal is output to the transducer driving circuit 32. The transducer driving circuit 32 receives the voltage signal, converts the voltage signal into a digital signal, and outputs the digital signal to the CPU 38.

The motor driving circuit 31 is a device for driving the linear motor 29 and a linear encoder 46. The linear encoder 46 is provided in the first support body 15, and the linear encoder 46 detects the position of the moving body 16. The motor driving circuit 31 receives an instruction signal from the CPU 38. Then, the position and the traveling speed of the moving body 16 are detected using the linear encoder 46. Then, the motor driving circuit 31 drives the linear motor 29 so that the moving body 16 is located at a position indicated by the instruction signal.

In addition to the rotary knob 10 or the keyboard 11, a device for performing cable communication and wireless communication with an external computer is included in the input device 12. Various kinds of data are input to the CPU 38 and the memory 41 through the input device 12. The operator inputs an instruction of the relative position of the moving body 16 with respect to the acoustic lens 13 by operating the input device 12.

The display device 9 is a display device, such as a liquid crystal display (LCD) or an organic light-emitting diode (OLED). Measurement conditions, the ultrasonic images to 37 that are measurement results, and the like are displayed on the display device 9.

The memory 41 is a concept including a semiconductor memory, such as a RAM or a ROM, or an external storage device, such as a hard disk or a DVD-ROM. Functionally, a storage region for storing program software 47, in which the control procedure of the operation of the ultrasonic measurement apparatus 1 is described, or a storage region for storing ultrasonic image data 48 detected by the ultrasonic element array 18 is set. In addition, a storage region for storing motor driving data 49, which is the data of conditions for driving the linear motor 29, is set. In addition, a storage region functioning as a temporary file or a work area for the CPU 38 or other various kinds of storage regions are set.

The CPU 38 moves the moving body 16 according to the program software 47 stored in the memory 41. Then, the CPU 38 performs control to detect the reflected wave by emitting the ultrasonic wave 24 from the ultrasonic element array 18. As a specific function realizing section, the CPU 38 has an ultrasonic wave reception and transmission control section 50. The ultrasonic wave reception and transmission control section 50 performs control to acquire the data of the ultrasonic wave 24 by making the transducer driving circuit 32 drive the ultrasonic element array 18.

In addition, the CPU 38 has a movement control section 51. The movement control section 51 receives information of the detection position input from the input device 12. The information of the detection position is information that the operator sets by operating the input device 12. Then, the movement control section 51 receives the position data of the moving body 16 detected by the linear encoder 46. Then, the movement control section 51 performs control to move the moving body 16 so that the moving body 16 is located at the position input by the operator. That is, the input device 12 receives an input of the relative position between the acoustic lens 13 and the ultrasonic element array 18 that are moved relatively each other by the linear motor 29. The movement control section 51 controls the relative position between the acoustic lens 13 and the ultrasonic element array 18.

In addition, the CPU 38 has an image forming section 52. The image forming section 52 receives digital data of the reflected waves output from the transducer driving circuit 32. Then, an ultrasonic image is formed by the data of reflected waves. In the present embodiment, each of the functions described above is realized by program software using the CPU 38. However, in a case where each of the functions described above can be realized by a single electronic circuit (hardware) in which the CPU 38 is not used, it is also possible to use such an electronic circuit.

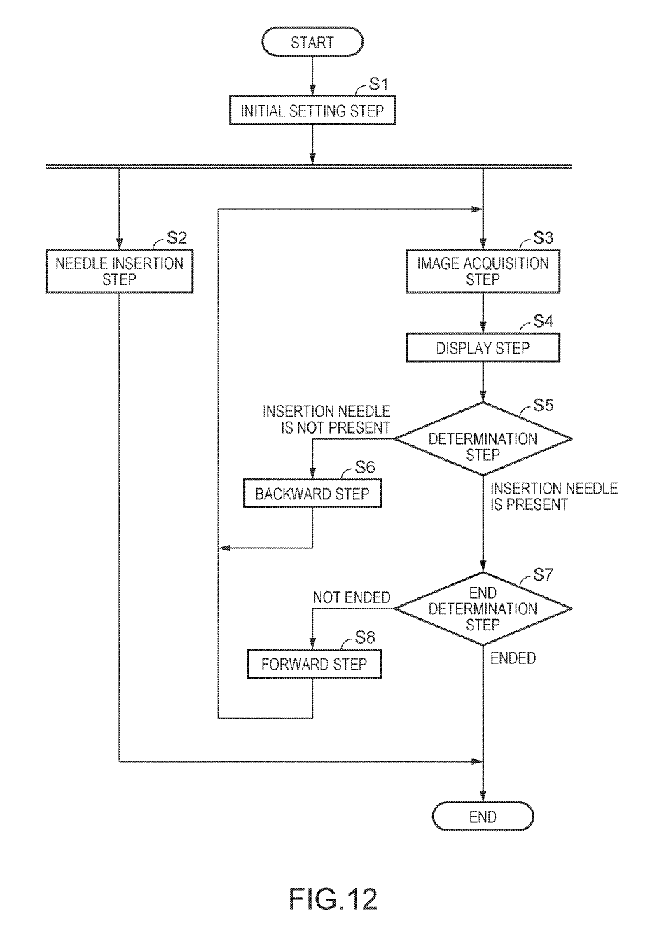

Next, an insertion needle insertion method for inserting the insertion needle 8a into the blood vessel 7 using the ultrasonic measurement apparatus 1 will be described with reference to FIG. 12. FIG. 12 is a flowchart of the insertion needle insertion method. The operation of the ultrasonic measurement apparatus 1 is performed by two operators of first and second operators.

In the flowchart shown in FIG. 12, step S1 is an initial setting step. This step is a step in which the movement control section 51 moves the moving body 16 to the end on the −Y direction side by making the motor driving circuit 31 drive the linear motor 29. Since the insertion needle 8a is inserted from the −Y direction side, the ultrasonic element array 18 emits the ultrasonic wave 24 to the −Y direction side and stands by. Then, the process proceeds to steps S2 and S3. Step S2 corresponds to a needle insertion step. This step is a step in which the first operator inserts the insertion needle 8a into the arm 6a. At this time, the first operator inserts the insertion needle 8a while observing the ultrasonic image displayed on the display device 9. Steps S2 and S3 to S6 are performed in parallel.

Step S3 is an image acquisition step. This step is a step of acquiring an ultrasonic image. First, the ultrasonic wave reception and transmission control section 50 emits the ultrasonic wave 24 into the arm 6a by making the transducer driving circuit 32 drive the ultrasonic element array 18. Some of the ultrasonic waves 24 are reflected by the blood vessel 7, the insertion needle 8a, or the like. Some of the reflected ultrasonic waves 24 reach the ultrasonic element array 18. In the ultrasonic element array 18, transducers are vibrated by the ultrasonic waves 24 that have reached the ultrasonic element array 18, thereby outputting a voltage proportional to the strength of the ultrasonic wave 24 to the transducer driving circuit 32. The transducer driving circuit 32 converts the voltage proportional to the strength of the ultrasonic wave 24 into digital data, and outputs the digital data to the image forming section 52 of the CPU 38. The image forming section 52 forms the ultrasonic image data 48 by combining the digital data, and stores the ultrasonic image data 48 in the memory 41. Then, the process proceeds to step S4.

Step S4 is a display step. This step is a step in which the display device 9 displays the ultrasonic image data 48. Then, the process proceeds to step S5. Step S5 is a determination step. This step is a step in which the second operator determines whether or not the ultrasonic image includes an insertion needle image while observing the display device 9. When the second operator determines that the ultrasonic image does not include an insertion needle image, the process proceeds to step S6. When the second operator determines that the ultrasonic image includes an insertion needle image, the process proceeds to step S7.

Step S6 is a backward step. This step is a step of moving the moving body 16 in the −Y direction. The second operator operates the input device 12 to input an instruction to move the moving body 16 by a predetermined distance in the −Y direction. The movement control section 51 receives a movement instruction signal, thereby receiving the position of the moving body 16 indicated by the linear encoder 46. When the moving body 16 is located at the end in the −Y direction, the position of the moving body 16 is maintained. Then, a message indicating that the ultrasonic wave 24 has reached the end in the −Y direction is displayed on the display device 9. When the moving body 16 is not located at the end in the −Y direction, the motor driving circuit 31 is made to drive the linear motor 29 so that the moving body 16 is moved by a predetermined distance in the −Y direction. Then, the process proceeds to step S3.

Step S7 is an end determination step. This step is a step of determining whether or not to end the measurement of the position of the insertion needle 8a. In this step, the second operator checks an ultrasonic image displayed on the display device 9. When the insertion needle 8a reaches the blood vessel 7 and the distal end portion 8b of the insertion needle 8a is not inserted into the blood vessel 7, it is determined that the measurement of the position of the insertion needle 8a is not ended. Then, the process proceeds to step S8. When the insertion needle 8a reaches the blood vessel 7 and the distal end portion 8b of the insertion needle 8a is inserted into the blood vessel 7, it is determined that the measurement of the position of the insertion needle 8a is ended. Then, the step of inserting the insertion needle 8a into the blood vessel 7 is ended.

Step S8 is a forward step. This step is a step of moving the moving body 16 in the +Y direction. The second operator operates the input device 12 to input an instruction to move the moving body 16 by a predetermined distance in the +Y direction. The movement control section 51 receives a movement instruction signal, thereby receiving the position of the moving body 16 indicated by the linear encoder 46. When the moving body 16 is located at the end in the +Y direction, the position of the moving body 16 is maintained. Then, a message indicating that the ultrasonic wave 24 has reached the end in the +Y direction is displayed on the display device 9. When the moving body 16 is not located at the end in the +Y direction, the motor driving circuit 31 is made to drive the linear motor 29 so that the moving body 16 is moved by a predetermined distance in the +Y direction. Then, the process proceeds to step S3.

Next, an insertion needle insertion method corresponding to steps S4 to S8 shown in FIG. 12 will be described in detail with reference to FIGS. 5 to 8. In the image acquisition step of step S3, as shown in FIG. 7, the insertion needle 8a is located on the −Y direction side with respect to the ultrasonic wave 24. At this time, the ultrasonic image 36 shown in FIG. 8 is displayed on the display device 9 in the display step of step S4. In the determination step of step S5, the second operator determines that the ultrasonic image 36 does not include an insertion needle image. Then, in the backward step of step S6, the second operator moves the moving body 16 in the −Y direction by operating the input device 12. The amount of movement of the moving body 16 is a predetermined short distance.

As a result, in step S3, as shown in FIG. 5, the ultrasonic wave 24 is emitted to the insertion needle 8a. Then, in step S4, the ultrasonic image 35 shown in FIG. 6 is displayed on the display device 9. The blood vessel image 35a and the insertion needle image 35b are displayed on the ultrasonic image 35. Since the amount of movement of the moving body 16 is short, the insertion needle image 35b of the location of the distal end portion 8b of the insertion needle 8a is displayed on the ultrasonic image 35. Through the ultrasonic image 35, the first operator can recognize that the blood vessel image 35a and the insertion needle image 35b are away from each other. Then, in step S2, the first operator further inserts the insertion needle 8a. As a result, the distal end portion 8b of the insertion needle 8a is located on the +Y direction side with respect to the location where the ultrasonic wave 24 passes.

In the determination step of step S5, the second operator determines that the ultrasonic image 35 includes the insertion needle image 35b. Then, in the forward step of step S8, the second operator moves the moving body 16 by a predetermined distance in the +Y direction by operating the input device 12. Thus, steps S2 to S8 are repeated. The first operator can advance the insertion needle 8a while checking the position of the distal end portion 8b of the insertion needle 8a according to this procedure. Then, as shown in FIG. 9, the insertion needle 8a reaches the blood vessel 7. In step S4, the first operator checks that the insertion needle 8a has reached the blood vessel 7, and further inserts the insertion needle 8a into the blood vessel 7. Then, the first and second operators check that the insertion needle 8a has been inserted into the blood vessel 7 by checking the display device 9. In the end determination step of step S7, determination to end a step of acquiring an ultrasonic image is made, and a step of inserting the insertion needle 8a into the blood vessel 7 is ended.

As described above, according to the present embodiment, the following effect is obtained.

(1) According to the present embodiment, the ultrasonic measurement apparatus 1 includes the ultrasonic element array 18, the acoustic lens 13, and the linear guide 23. The ultrasonic element array 18 emits the ultrasonic wave 24 in the first direction 25. The acoustic lens 13 is a columnar lens, and changes the traveling direction of the ultrasonic wave 24 emitted from the ultrasonic element array 18. The acoustic lens 13 has a shape in which a predetermined sectional shape extends in the column axis direction 27. When the acoustic lens 13 and the ultrasonic element array 18 move relatively each other, the linear guide 23 restricts the movement direction so that the acoustic lens 13 and the ultrasonic element array 18 move relatively each other in the second direction 26.

Accordingly, the second direction 26 in which the acoustic lens 13 and the ultrasonic element array 18 move relatively each other is a direction crossing the first direction 25. The second direction 26 is a direction crossing the column axis direction 27 of the acoustic lens 13. The acoustic lens 13 is a lens whose thickness in the first direction 25 changes corresponding to the position of the second direction 26. That is, the surface of the acoustic lens 13 in the first direction 25 is a convex shape. The ultrasonic wave 24 is input to the acoustic lens 13, and is output from the acoustic lens 13. The traveling direction of the ultrasonic wave 24 output from the acoustic lens 13 changes corresponding to the angle of the surface of the acoustic lens 13 on the first direction 25 side.

The acoustic lens 13 and the ultrasonic element array 18 move relatively each other in the second direction 26. Accordingly, since the angle of the surface on the first direction 25 side of the location where the ultrasonic wave 24 is output from the acoustic lens 13 is changed, the traveling direction of the ultrasonic wave 24 is changed when the ultrasonic wave 24 is output from the acoustic lens 13. Therefore, the ultrasonic measurement apparatus 1 can change the traveling direction of the ultrasonic wave 24. The traveling direction of the ultrasonic wave 24 changes only in the second direction 26, but does not change in the column axis direction 27. Since the traveling direction of the ultrasonic wave 24 does not change in the column axis direction 27, it is possible to suppress the shaking of the ultrasonic image displayed on the display device 9 in the horizontal direction. Therefore, the ultrasonic measurement apparatus 1 can change the traveling direction of the ultrasonic wave 24 with good operability.

(2) According to the present embodiment, the acoustic lens 13 is a convex lens. In addition, the acoustic lens 13 is used in contact with the arm 6a. The material of the acoustic lens 13 is a material in which the traveling speed of the ultrasonic wave 24 is higher than the traveling speed of the ultrasonic wave 24 in the arm 6a. For this reason, the traveling speed of the ultrasonic wave 24 is reduced when the ultrasonic wave 24 enters the subject under examination from the acoustic lens 13. In addition, since the acoustic lens 13 is a convex lens and the arm 6a functions as a concave lens, the ultrasonic measurement apparatus 1 can emit the ultrasonic wave 24 to a place wider than the width of the acoustic lens in the second direction 26.

(3) According to the present embodiment, the ultrasonic measurement apparatus 1 further includes the linear motor 29, the movement control section 51, and the input device 12. The linear motor 29 moves the acoustic lens 13 and the ultrasonic element array 18 relatively each other. The movement control section 51 controls the relative position between the acoustic lens 13 and the ultrasonic element array 18. The second operator inputs the relative position using the input device 12. Then, the input device 12 receives an input of the relative position between the acoustic lens 13 and the ultrasonic element array 18 that are moved relatively each other by the linear motor 29. The movement control section 51 controls the linear motor 29 to move the acoustic lens 13 and the ultrasonic element array 18 relatively each other. Therefore, the second operator can perform an operation of moving the acoustic lens 13 and the ultrasonic element array 18 in the second direction 26. As a result, the distal end portion 8b of the insertion needle 8a can be displayed on the display device 9.

(4) According to the present embodiment, the gel 14 is disposed between the ultrasonic element array 18 and the acoustic lens 13. A change in the acoustic impedance between the ultrasonic element array 18 and the acoustic lens 13 can be reduced by the gel 14. As a result, it is possible to suppress the reflection of the ultrasonic wave 24 between the ultrasonic element array 18 and the acoustic lens 13.

(5) According to the present embodiment, since the first operator can observe the distal end portion 8b of the insertion needle 8a in a clear ultrasonic image, it is possible to reduce the failure of insertion. In addition, it is possible to reduce complications caused by blood leakage due to the failure of insertion.

### Second Embodiment

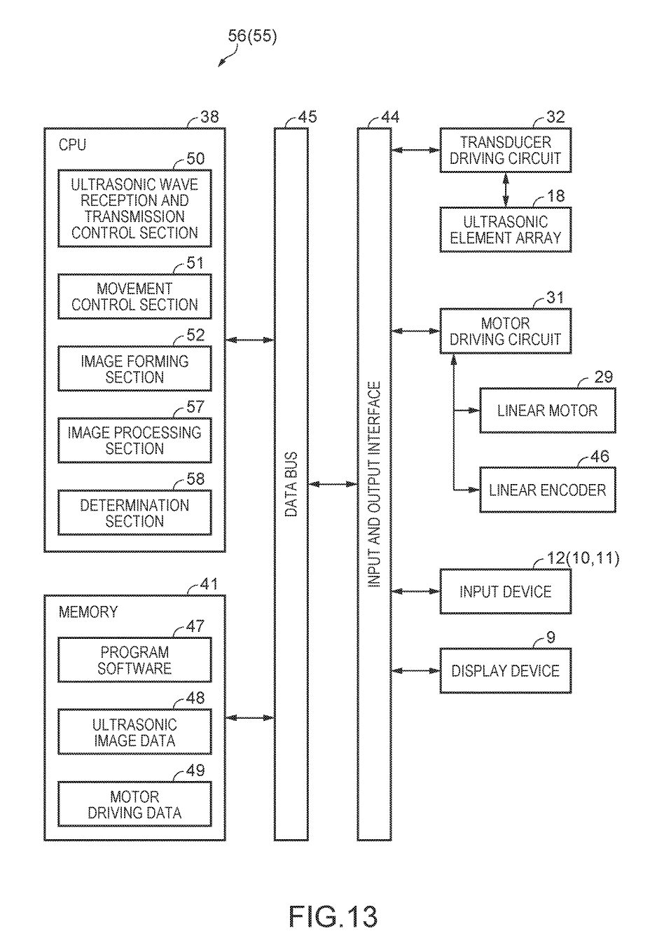

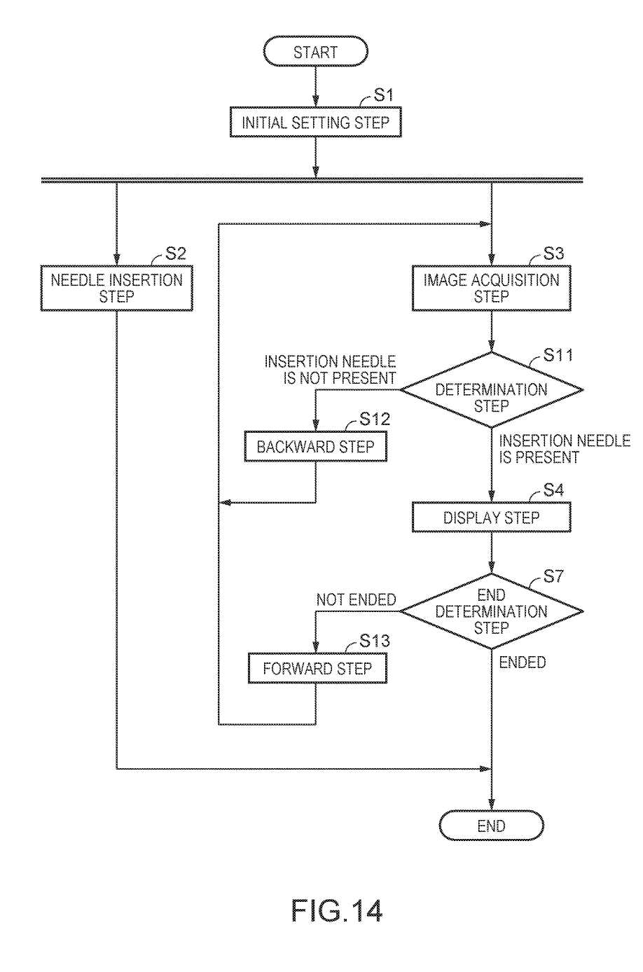

Next, an embodiment of the ultrasonic measurement apparatus will be described with reference to FIGS. 13 and 14. The present embodiment is different from the first embodiment in that the CPU 38 has functions of an image processing section and a determination section. In addition, the explanation of the same points as in the first embodiment will be omitted.

FIG. 13 is an electric control block diagram of the ultrasonic measurement apparatus. That is, in the present embodiment, as shown in FIG. 13, in a control device 56 as a control unit of the ultrasonic measurement apparatus 55, the CPU 38 has an image processing section 57 as a specific function realizing section. The image processing section 57 performs image processing on the ultrasonic image formed by the image forming section 52. First, the image processing section 57 removes noise from the ultrasonic image. This makes the brightness of a portion having a minute area the same as the surrounding brightness. Then, division into a bright portion and a dark portion is performed using a predetermined brightness setting value. Usually, this step is called binarization. Then, the respective bright and dark regions are divided into separate islands, and the number of pixels of each island is measured. This island indicates a portion when a bright portion and a dark portion are separated from each other and each region of a region of a bright portion and a region of a dark portion. The number of pixels is a feature quantity corresponding to the area of the ultrasonic image. In addition, the image processing section 57 calculates the feature quantity of each island by calculating the circumference and the width of each island and performing classification as to whether or not the island has a ring shape.

The CPU 38 has a determination section 58. The determination section 58 performs classification into an island corresponding to a blood vessel image and an island corresponding to an insertion needle image from the feature quantity, such as the area measured by the image processing section 57. Then, the determination section 58 checks an insertion needle image in the ultrasonic image to determine whether or not the ultrasonic image includes an insertion needle image. The motor driving data 49 of the memory 41 includes data of the amount of movement for moving the moving body 16 in the forward step and the backward step. The determination section 58 has a function of outputting an instruction signal, which indicates whether to advance the moving body 16 in the +Y direction or to retreat the moving body 16 in the −Y direction according to a determination result regarding whether or not the ultrasonic image includes an insertion needle image, to the movement control section 51. The movement control section 51 receives the instruction signal of the determination section 58, and performs control to move the moving body 16 based on the instruction signal.

FIG. 14 is a flowchart of the insertion needle insertion method. The operation of the ultrasonic measurement apparatus 55 is performed by one first operator. Since steps S1 to S3 are the same as in the first embodiment, the explanation thereof will be omitted. After step S3, a determination step of step S11 is performed. In step S11, the CPU 38 determines whether or not the ultrasonic image 36 includes an insertion needle image. Specifically, the image processing section 57 calculates a feature quantity, such as the area of each island of an image included in the ultrasonic image. The insertion needle image has a vertically long shape, and the blood vessel image has a circular shape. Such determination can be made based on the ratio of the horizontal width and the vertical length. Since the area of the insertion needle image is smaller than the area of the blood vessel image, such determination can be made by comparison with a predetermined determination value. In addition, respective islands are classified into an island corresponding to the blood vessel image and an island corresponding to the insertion needle image. Then, the determination section 58 checks an insertion needle image in the ultrasonic image to determine whether or not the ultrasonic image includes an insertion needle image.

When the determination section 58 determines that the ultrasonic image does not include an insertion needle image in the determination step of step S11, the process proceeds to the backward step of step S12. Then, in the backward step of step S12, the movement control section 51 controls the motor driving circuit 31 to drive the linear motor 29 so that the moving body 16 is moved in the −Y direction. Data of the amount of movement as the amount of relative movement by which the moving body 16 moves is stored in the motor driving data 49, and is a predetermined length. Then, the process proceeds to step S3. When the ultrasonic image does not include an insertion needle image, steps S3, S11, and S12 are repeated so that the ultrasonic image includes an insertion needle image. Then, the control device 56 controls the moving body 16 so that the ultrasonic image includes an insertion needle image.

When the determination section 58 determines that the ultrasonic image includes an insertion needle image in the determination step of step S11, the process proceeds to the display step of step S4. In step S4, the ultrasonic image is displayed on the display device 9. The insertion needle 8a is a rod-shaped member having the distal end portion 8b, and the determination section 58 proceeds to step S4 only when an insertion needle image is detected in the ultrasonic image. Then, in step S4, an ultrasonic image including the insertion needle image of the distal end portion is displayed on the display device 9. Since the ultrasonic image includes the insertion needle image, the operator can check the position of the insertion needle. Then, the process proceeds to the end determination step of step S7. When the first operator determines “not ended”, the process proceeds to the forward step of step S13. In step S13, the movement control section 51 controls the motor driving circuit 31 to drive the linear motor 29 so that the moving body 16 is moved in the +Y direction. Data of the amount of movement as the amount of relative movement by which the moving body 16 moves is stored in the motor driving data 49, and is a predetermined length. Then, the process proceeds to step S3.

When the first operator determines “ended” in the end determination step of step S7, the step of inserting the insertion needle 8a into the blood vessel 7 is ended.

An ultrasonic image is displayed on the display device 9 only when the moving body 16 is moved in the +Y direction in the forward step of step S13 and the ultrasonic image includes an insertion needle image in the determination step of step S11. When the ultrasonic wave 24 is emitted to the +Y direction side from the distal end portion 8b of the insertion needle 8a, no ultrasonic image is displayed on the display device 9 since the ultrasonic image does not include an insertion needle image. Therefore, an ultrasonic image including the image of the distal end portion 8b of the insertion needle 8a is displayed on the display device 9.

As described above, according to the present embodiment, the following effect is obtained.

(1) According to the present embodiment, the image forming section 52 forms an ultrasonic image from the data of reflected waves, and the determination section 58 determines an insertion needle image of the ultrasonic image. At this time, when the ultrasonic image does not include an insertion needle image, the movement control section 51 controls the linear motor 29. Then, the ultrasonic image is made to include an insertion needle image. As a result, the first operator can recognize the position of the distal end portion 8b of the insertion needle 8a by observing the ultrasonic image.

(2) According to the present embodiment, the insertion needle 8a is a rod-shaped member having the distal end portion 8b. The determination section 58 detects the distal end portion 8b depending on whether or not the distal end portion 8b is reflected on the ultrasonic image. Then, the ultrasonic measurement apparatus 55 displays the ultrasonic image including the distal end portion 8b on the display device 9. Therefore, the first operator can check the position of the distal end portion 8b even when the distal end portion 8b of the insertion needle 8a moves.

### Third Embodiment

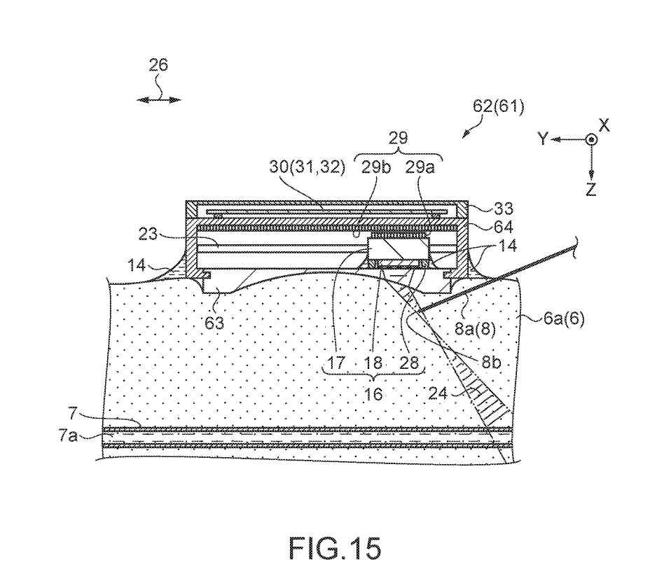

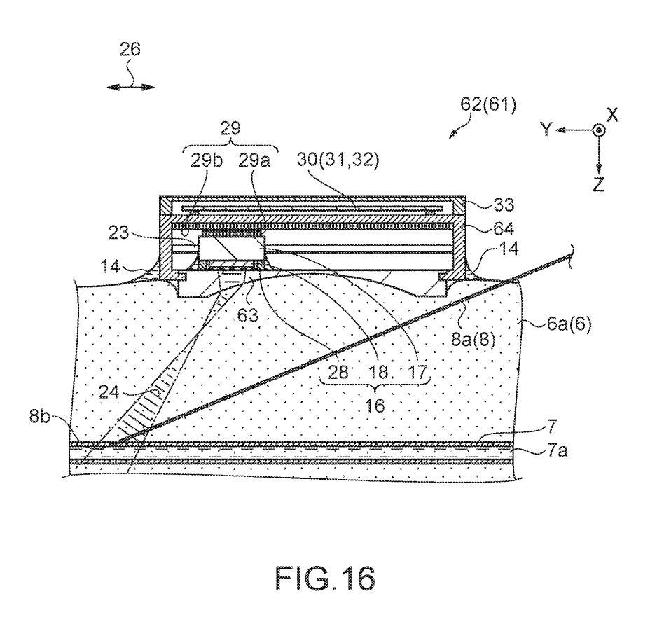

Next, an embodiment of the ultrasonic measurement apparatus will be described with reference to FIGS. 15 and 16. The present embodiment is different from the first embodiment in that the acoustic lens is a concave lens. In addition, the explanation of the same points as in the first embodiment will be omitted.

FIGS. 15 and 16 are schematic side sectional views showing the structure of an ultrasonic probe. FIG. 15 is a diagram when the moving body 16 has moved in the −Y direction, and FIG. 16 is a diagram when the moving body 16 has moved in the +Y direction. That is, in the present embodiment, as shown in FIG. 15, an ultrasonic probe 62 provided in an ultrasonic measurement apparatus 61 includes an acoustic lens 63. The acoustic lens 63 is a concave lens. The acoustic lens 63 is pressed against the skin of the arm 6a, and is used in contact with the skin of the arm 6a.

A bottomed rectangular tubular first support body 64 is provided on the -Z direction side of the acoustic lens 63. A moving body 16 that moves in the Y direction is provided in the first support body 64. The moving body 16 includes a substrate 17, and an ultrasonic element array 18 as an ultrasonic wave transmission unit and an ultrasonic wave receiving unit is provided on the surface of the substrate 17 on the +Z direction side.

When the moving body 16 moves to the −Y direction side along the second direction 26, the ultrasonic wave 24 travels to the −Y direction side due to the acoustic lens 63. The material of the acoustic lens 63 is a material in which the traveling speed of the ultrasonic wave 24 is lower than the traveling speed of the ultrasonic wave 24 in the arm 6a. The traveling speed of the ultrasonic wave 24 in the arm 6a is 1530 m/s. Silicone rubber and PDMS can be used as materials of the acoustic lens 63. The traveling speed of the ultrasonic wave 24 in silicone rubber is 1000 m/s, and the traveling speed of the ultrasonic wave 24 in polydimethylsiloxane (PDMS) is 900 to 10000 m/s. In the present embodiment, for example, silicone rubber is used as a material of the acoustic lens 63.

At this time, the traveling speed of the ultrasonic wave 24 is increased when the ultrasonic wave 24 enters the arm 6a from the acoustic lens 63. The acoustic lens 63 is a concave lens, and the subject under examination functions as a convex lens. Accordingly, the ultrasonic probe 62 can emit the ultrasonic wave 24 to a place wider than the width of the acoustic lens 63 in the second direction 26. By moving the moving body 16 to the −Y direction side, the ultrasonic wave 24 is made to travel to the −Y direction side. Therefore, it is possible to detect the insertion needle 8a inserted from the −Y direction side.

As shown in FIG. 16, the insertion needle 8a is inserted in the +Y direction. At this time, the control device 3 moves the moving body 16 to the +Y direction side along the second direction 26. The ultrasonic wave 24 travels to bend in the +Y direction due to the acoustic lens 63. Then, since the ultrasonic wave 24 passes through the distal end portion 8b of the insertion needle 8a, the ultrasonic element array 18 detects reflected waves from the distal end portion 8b of the insertion needle 8a.

As described above, according to the present embodiment, the following effect is obtained.

(1) According to the present embodiment, the acoustic lens 63 is a concave lens. In addition, the acoustic lens 63 is used in contact with the arm 6a. The material of the acoustic lens 63 is a material in which the traveling speed of the ultrasonic wave 24 is lower than the traveling speed of the ultrasonic wave 24 in the arm 6a. Accordingly, the traveling speed of the ultrasonic wave 24 is increased when the ultrasonic wave 24 enters the arm 6a from the acoustic lens 63. In addition, since the acoustic lens 63 is a concave lens, the ultrasonic measurement apparatus 61 can emit the ultrasonic wave 24 to a place wider than the width of the acoustic lens 63 in the second direction 26.

### Fourth Embodiment

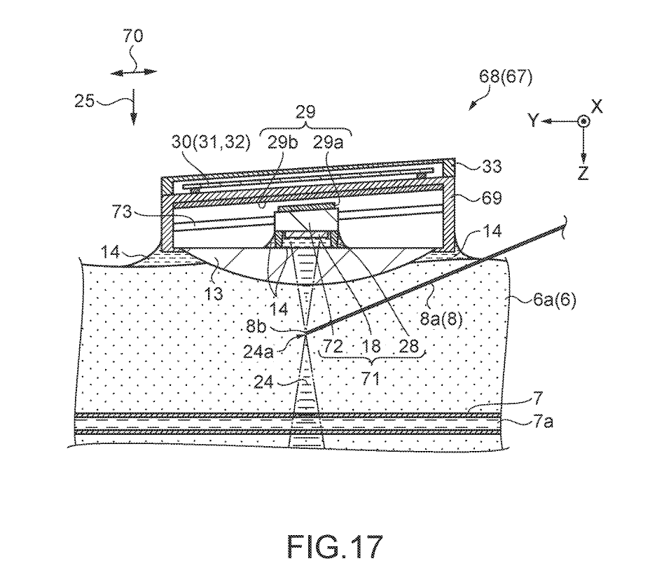

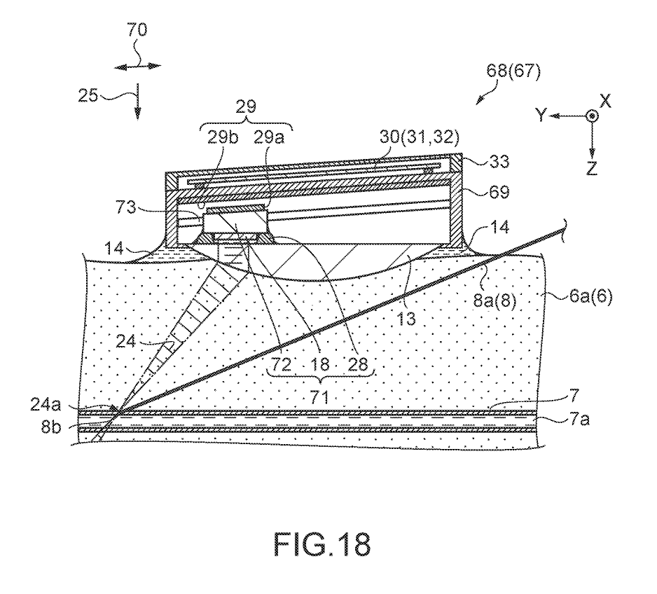

Next, an embodiment of the ultrasonic measurement apparatus will be described with reference to FIGS. 17 and 18. The present embodiment is different from the first embodiment in that the moving body 16 moves obliquely with respect to the first direction 25. In addition, the explanation of the same points as in the first embodiment will be omitted.

FIGS. 17 and 18 are schematic side sectional views showing the structure of an ultrasonic probe. FIG. 17 is a diagram when the moving body has moved to the center, and FIG. 18 is a diagram when the moving body has moved in the +Y direction. That is, in the present embodiment, as shown in FIG. 17, an ultrasonic probe 68 provided in an ultrasonic measurement apparatus 67 includes an acoustic lens 13. The acoustic lens 13 is pressed against the skin of the arm 6a, and is used in contact with the skin of the arm 6a.

A bottomed rectangular tubular first support body 69 is provided on the −Z direction side of the acoustic lens 13. A moving body 71 that moves along an oblique second direction 70 crossing the first direction 25 is provided inside the first support body 69. The moving body 71 includes a substrate 72, and an ultrasonic element array 18 is provided on the surface of the substrate 72 on the +Z direction side.

A linear guide 73 as a movement restriction unit is provided on both sides of the substrate 72 on the +X and −X direction sides. The moving body 71 is moved in the second direction 70 by the linear guide 73. When the moving body 71 moves in the second direction 70, the distance between the moving body 71 and the acoustic lens 13 is changed. The packing 28 has elasticity, and expands and contracts in the first direction 25. Therefore, the gel 14 is held between the ultrasonic element array 18 and the acoustic lens 13.

As shown in FIG. 18, the insertion needle 8a is inserted in the +Y direction. At this time, the control device 3 moves the moving body 71 to the +Y direction side along the second direction 70. The ultrasonic wave 24 travels to bend in the +Y direction due to the acoustic lens 13. Then, since the ultrasonic wave 24 passes through the distal end portion 8b of the insertion needle 8a, the ultrasonic element array 18 detects reflected waves from the distal end portion 8b of the insertion needle 8a.

When the moving body 71 moves along the second direction 70, the position of the moving body 71 in the first direction 25 with respect to the arm 6a is changed. A place where the ultrasonic wave 24 concentrates due to the acoustic lens 13 is defined as a focal point 24a. When the moving body 71 moves along the second direction 70, the position of the focal point 24a in the first direction 25 with respect to the arm 6a is changed. When the distal end portion 8b of the insertion needle 8a is located on the −Y direction side, the distal end portion 8b is located in a shallow place of the arm 6a. Then, when the moving body 71 is located on the −Y direction side, the focal point 24a is also located in a shallow place of the arm 6a. In a place close to the focal point 24a, it is possible to make an ultrasonic image clear compared with a place far from the focal point 24a. Therefore, it is possible to make an insertion needle image corresponding to the distal end portion 8b clear.

When the distal end portion 8b of the insertion needle 8a is located on the +Y direction side, the distal end portion 8b is located in a deep place of the arm 6a. Then, when the moving body 71 is located on the +Y direction side, the focal point 24a is also located in a deep place of the arm 6a. Therefore, it is possible to make an insertion needle image corresponding to the distal end portion 8b clear.

As described above, according to the present embodiment, the following effect is obtained.

(1) According to the present embodiment, since the ultrasonic probe 68 changes the position of the focal point 24a according to the movement of the moving body 71, an image corresponding to the distal end portion 8b of the insertion needle 8a in an ultrasonic image can be made clear.

### Fifth Embodiment

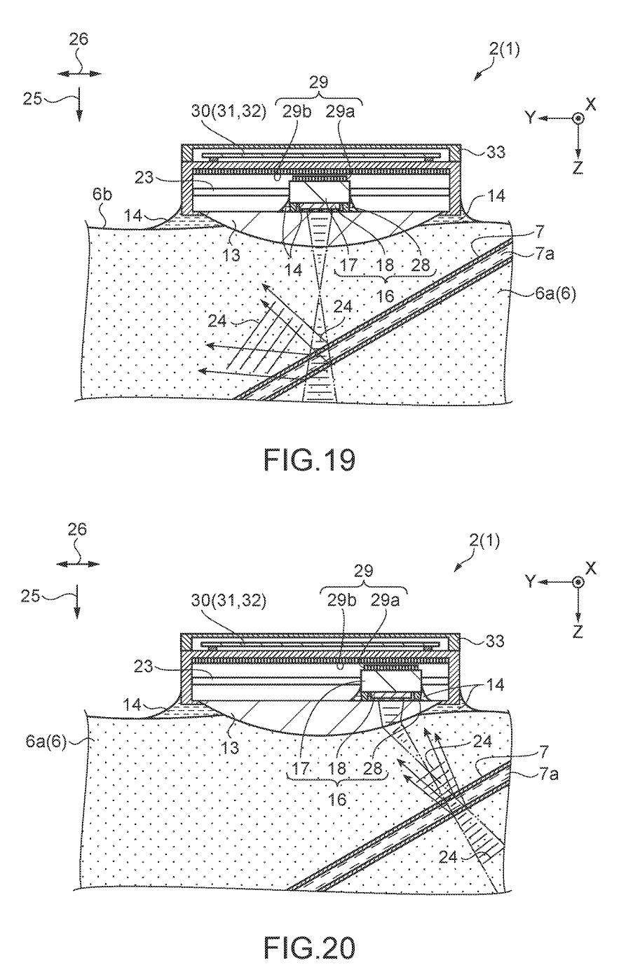

Next, an embodiment using an ultrasonic measurement apparatus will be described with reference to FIGS. 19 and 20. The present embodiment is different from the first embodiment in that the blood vessel 7 that is obliquely located in the arm 6a is imaged. In addition, the explanation of the same points as in the first embodiment will be omitted.

FIGS. 19 and 20 are schematic side sectional views for explaining a method of detecting a blood vessel using ultrasonic waves. FIG. 19 is a diagram when the moving body has moved to the center, and FIG. 20 is a diagram when the moving body has moved in the −Y direction. That is, in the present embodiment, as shown in FIG. 19, the blood vessel 7 is disposed obliquely with respect to a surface 6b of the arm 6a. In the second direction 26, the moving body 16 is located at the center of the ultrasonic probe 2. At this time, the ultrasonic wave 24 emitted from the ultrasonic element array 18 passes through the acoustic lens 13 to travel in the first direction 25.

The ultrasonic wave 24 emitted to the blood vessel 7 is reflected by the blood vessel 7. Since the blood vessel 7 is oblique with respect to the first direction 25, a main portion of the reflected wave of the ultrasonic wave 24 travels obliquely with respect to the first direction 25. Then, the strength of the reflected wave of the ultrasonic wave 24 traveling in the first direction 25 becomes low. Therefore, the strength of the ultrasonic wave 24 received by the ultrasonic element array 18 is low. For this reason, in the ultrasonic image displayed on the display device 9 by the ultrasonic measurement apparatus 1, an image corresponding to the blood vessel 7 becomes thin. Accordingly, it is difficult to check the image. In particular, since the strength of the reflected wave is low on the inner wall of the blood vessel 7, it is difficult to check an image corresponding to the inner wall of the blood vessel 7 in the ultrasonic image.

As shown in FIG. 20, when the moving body 16 moves in the −Y direction, the ultrasonic wave 24 emitted from the ultrasonic element array 18 passes through the acoustic lens 13 to travel in the −Y direction and +Z direction. The ultrasonic wave 24 emitted to the blood vessel 7 is reflected by the blood vessel 7. The traveling direction of the ultrasonic wave 24 and the direction in which the blood vessel 7 extends are approximately perpendicular to each other. At this time, a main portion of the reflected wave of the ultrasonic wave 24 travels toward the ultrasonic element array 18. Then, the strength of the reflected wave of the ultrasonic wave 24 traveling toward the ultrasonic element array 18 becomes high. Accordingly, the strength of the ultrasonic wave 24 received by the ultrasonic element array 18 is high. For this reason, in the ultrasonic image displayed on the display device 9 by the ultrasonic measurement apparatus 1, an image corresponding to the blood vessel 7 becomes clear. Accordingly, it is easy to check the image. Also on the inner wall of the blood vessel 7, the strength of the reflected wave is high. Therefore, it is easy to check the image corresponding to the blood vessel 7 in the ultrasonic image.

As described above, according to the present embodiment, the following effect is obtained.

(1) According to the present embodiment, the ultrasonic probe 2 can change the traveling direction of the ultrasonic wave 24 according to the movement of the moving body 16. Even when the blood vessel 7 is oblique with respect to the first direction 25, the image corresponding to the blood vessel 7 can be made clear by changing the traveling direction of the ultrasonic wave 24 by moving the moving body 16.

The invention is not limited to the embodiments described above, and various modifications or improvements can be made within the technical idea of the invention by those skilled in the art. Modification examples will be described below.

### Modification Example 1

In the first embodiment described above, the linear motor 29 moves the moving body 16. Without being limited thereto, an operator may press the moving body 16 with a finger. Since the linear motor 29 and the motor driving circuit 31 are not necessary, it is possible to manufacture the ultrasonic measurement apparatus 1 with good productivity. When using a motor, motors other than the linear motor 29 may be used. A ball screw and a motor having a rotary shaft may be combined. Alternatively, a piezoelectric motor may be used.

### Modification Example 2

In the first embodiment described above, the ultrasonic probe 2 and the control device 3 are connected to each other by the wiring line 4. The ultrasonic probe 2 and the control device 3 may wirelessly communicate with each other. It is possible to easily provide the control device 3 in a place where the display device 9 is easy to see.

### Modification Example 3

In the first embodiment described above, an example of injection into the blood vessel 7 is shown. In addition, at the time of injection into nerves or at the time of injection into an organ, the ultrasonic measurement apparatus 1 may be used. Also at this time, it is possible to accurately insert the insertion needle 8a to the target location. In addition, it is possible to suppress complications caused by damaging the nerve. In addition, when sampling the tissue of a lesion part for biological tissue diagnosis, the ultrasonic measurement apparatus 1 may be used. In addition, the ultrasonic measurement apparatus 1 maybe used for percutaneous radiofrequency ablation therapy. In the percutaneous radiofrequency ablation therapy, the electrode needle is inserted into the center of the tumor through the skin. Then, the current of the wavelength of the radio wave is applied to generate heat around the needle. The tumor is necrotized by the heat. The electrode needle is inserted to the center of the tumor while observing the tumor in the ultrasonic image formed by the ultrasonic measurement apparatus 1. Therefore, it is possible to locate the electrode needle with high positional accuracy.

The entire disclosure of Japanese Patent Application NO. 2016-041857 filed on Mar. 4, 2016 is expressly incorporated by reference herein.

What is claimed is:

1. An ultrasonic measurement apparatus, comprising:
an ultrasonic wave transmission unit that emits an ultrasonic wave in a first direction;
a columnar acoustic lens that changes a traveling direction of the ultrasonic wave emitted from the ultrasonic wave transmission unit; and
a movement restriction unit that restricts a movement direction such that the acoustic lens and the ultrasonic wave transmission unit move relatively each other in a second direction crossing the first direction and a column axis direction of the acoustic lens.

2. The ultrasonic measurement apparatus according to claim 1,
wherein the acoustic lens is a convex lens, and
a material of the acoustic lens is a material in which a traveling speed of an ultrasonic wave is higher than that of an ultrasonic wave in a subject under examination.

3. The ultrasonic measurement apparatus according to claim 1,
wherein the acoustic lens is a concave lens, and
a material of the acoustic lens is a material in which a traveling speed of an ultrasonic wave is lower than that of an ultrasonic wave in a subject under examination.

4. The ultrasonic measurement apparatus according to claim 1, further comprising:
a moving unit that moves the acoustic lens and the ultrasonic wave transmission unit relatively each other;
a control unit that controls a relative position between the acoustic lens and the ultrasonic wave transmission unit; and
an input unit that receives an input of the relative position.

5. The ultrasonic measurement apparatus according to claim 1, further comprising:
a moving unit that moves the acoustic lens and the ultrasonic wave transmission unit relatively each other;
a control unit that controls the moving unit;
an ultrasonic wave receiving unit that receives a reflected wave reflected by a subject under examination;
an image forming unit that forms an ultrasonic image from data of the reflected wave;
a determination unit that determines a subject of the ultrasonic image; and
a display unit that displays the ultrasonic image,
wherein, when the ultrasonic image does not include the subject, the control unit controls the moving unit such that the ultrasonic image includes the subject.

6. The ultrasonic measurement apparatus according to claim 5,
wherein the subject is a rod-shaped member having a distal end portion, and
the control unit detects the distal end portion, and displays the ultrasonic image including the distal end portion on the display unit.

7. The ultrasonic measurement apparatus according to claim 1,
wherein a liquid material is disposed between the ultrasonic wave transmission unit and the acoustic lens.

8. An ultrasonic probe, comprising:
an ultrasonic wave transmission unit that emits an ultrasonic wave in a first direction;
a columnar acoustic lens that changes a traveling direction of the ultrasonic wave emitted from the ultrasonic wave transmission unit; and
a movement restriction unit that restricts a movement direction such that the acoustic lens and the ultrasonic wave transmission unit move relatively each other in a second direction crossing the first direction and a column axis direction of the acoustic lens.
